(19) United States
(12) Patent Application Publication
(10) Pub. No.: US 2023/0238019 A1
(43) Pub. Date: Jul. 27, 2023

(54) MACHINE LEARNING MODELS FOR AUTOMATED PROCESSING OF AUDIO WAVEFORM DATABASE ENTRIES

(71) Applicant: Evernorth Strategic Development, Inc., St. Louis, MO (US)

(72) Inventors: Jonathan J. Lisic, Blacksburg, VA (US); John Ciliberti, Sparta, NJ (US)

(21) Appl. No.: 17/580,748

(22) Filed: Jan. 21, 2022

(51) Int. Cl. G10L 25/66; G10L 25/30; G10L 25/54

(52) U.S. Cl. CPC G10L 25/66; G10L 25/30; G10L 25/54

(57) ABSTRACT

A computer system includes memory hardware and processor hardware configured to execute stored instructions. The instructions include training a machine learning model with the historical feature vector inputs including multiple audio data entries and multiple claims data entries, to generate a condition likelihood output indicative of a specified condition associated with one of multiple historical database entities. The instructions include for each of a set of multiple database entities, generating a feature vector input according to audio data and the claims data associated with the entity, processing the feature vector input with the machine learning model to generate the condition likelihood output, and assigning the database entity to an identified condition subset in response to determining that the condition likelihood output is greater than a specified likelihood threshold. The instructions include transforming a user interface to display the condition likelihood output associated with the database entity.

### FIELD

The present disclosure relates to machine learning models for automated processing of audio waveform database entries.

### BACKGROUND

Audio data may be analyzed for detection of health conditions such as depression and anxiety. A neural network may be built using features that are tuned specifically for detection of a particular disease. Speech-to-text applications may utilize either a time series of raw waveform audio, or features generated from the spectral domain, for analyzing user speech data.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A computer system includes memory hardware configured to store a machine learning model, historical feature vector inputs, and computer-executable instructions, wherein the historical feature vector inputs include historical data structures specific to multiple historical database entities, and wherein the historical data structures include multiple audio data entries and multiple claims data entries. The system includes processor hardware configured to execute the instructions. The instructions include training the machine learning model with the historical feature vector inputs, including the multiple audio data entries and multiple claims data entries, to generate a condition likelihood output, wherein the condition likelihood output is indicative of a specified condition associated with one of the multiple historical database entities. The instructions include obtaining a set of multiple database entities, and for each database entity in the set of multiple database entities, obtaining audio data associated with the database entity, obtaining claims data associated with the database entity, generating a feature vector input according to the audio data and the claims data, processing, by the machine learning model, the feature vector input to generate the condition likelihood output, determining whether the condition likelihood output is greater than a specified likelihood threshold, and assigning the database entity to an identified condition subset of the multiple database entities in response to determining that the condition likelihood output is greater than the specified likelihood threshold. The instructions include, for each database entity in the identified condition subset, transforming a user interface to display the condition likelihood output associated with the database entity.

In other features, the memory hardware is configured to store multiple machine learning models each associated with a different one of multiple condition classification types, and the instructions include identifying one of the multiple machine learning models according to a specified condition prediction type, and processing the feature vector input includes processing the feature vector input using the selected machine learning model. In other features, the machine learning model includes a historic machine learning model, and training the historic machine learning model includes obtaining call transcription data associated with the multiple audio data entries, processing the call transcription data to using at least one of keyword and natural language processing to generate processed transcription input features, parsing the multiple audio data entries to define individual words, processing the defined individual words to generate processed audio input features, and supplying a training feature vector input to the machine learning model to train the machine learning model, wherein the training feature vector input includes the processed transcription input features and the processed audio input features.

In other features, the processed audio input features include at least one of an intensity of the audio waveform, a fundamental frequency, a formant frequency, Mel Frequency Cepstrum Coefficients (MFCCs), a glottal flow, a jitter value, a zero crossing value, a trailing intensity, and a white space length. In other features, the machine learning model comprises a raw audio model, and the raw audio model comprises a one-dimensional convolution layer which receives an input of multiple frames, wherein the convolution layer includes multiple filters, a recurrent layer which uses the input from the convolution layer to identify temporal dependence through use of a Long Short-Term Memory (LSTM) layer, and a final layer that maps the convolution layer and the recurrent layer to a final output.

In other features, the machine learning model comprises a processed audio model, and training the processed audio model includes separating the multiple audio data entries into temporal frames with overlap, for each temporal frame, obtaining multiple processed audio features, wherein the multiple processed audio features include at least one of log-Mel bank features associated with the frame, Mel Frequency Cepstrum Coefficients associated with the frame, MFCC summary statistics associated with the frame, and MFCC difference values between a current temporal index and a prior temporal index, and supplying a training feature vector input to the processed audio model to train the processed audio model, wherein the training feature vector input includes the multiple processed audio features. In other features, the specified condition associated with one of the multiple historical database entities includes at least one of a post-partum depression medical condition, an anxiety medical condition, a drug addiction medical condition, a Parkinson's disease medical condition, and a respiratory disorder medical condition.

In other features, the instructions include identifying a date associated with each of the multiple audio data entries and the multiple claims data entries, determining a date associated with a condition of each of the multiple historical database entities, based at least in part on the dates associated with the multiple claims data entries, and building a training dataset for training the machine learning model, wherein each audio data entry included in the training dataset has a date that is within a specified time window of the determined date associated with the condition of a corresponding one of the multiple historical database entities. In other features, training the machine learning model includes obtaining multiple social media data entries each associated with one of the multiple historical database entities, and generating one or more social media input features based on the multiple social media data entries, wherein the historical feature vector inputs include the one or more social media input features.

In other features, training the machine learning model includes comparing multiple condition likelihood outputs of the machine learning model to the historical data structures, determining whether an accuracy of the comparison is greater than or equal to a specified accuracy threshold, adjusting parameters of the machine learning model to retrain the machine learning model, in response to the accuracy of the comparison being less than the specified accuracy threshold, and saving the machine learning model for use in generating condition likelihood outputs, in response to the accuracy of the comparison being greater than or equal to the specified accuracy threshold. In other features, training the machine learning model includes separating portions of the historical feature vector inputs into structured training data and structured test data, training the machine learning model using the structured training data, testing the trained machine learning model using the structured test data, evaluating results of testing the trained machine learning model, and saving the machine learning model for use in generating condition likelihood outputs, in response to an accuracy of the evaluated results being greater than or equal to a specified accuracy threshold.

A computerized method for automated processing of audio waveform database entries using a machine learning model includes training a machine learning model with the historical feature vector inputs to generate a condition likelihood output, wherein the historical feature vector inputs include historical data structures specific to multiple historical database entities, wherein the historical data structures include multiple audio data entries and multiple claims data entries, and wherein the condition likelihood output is indicative of a specified condition associated with one of the multiple historical database entities. The method includes obtaining a set of multiple database entities, and for each database entity in the set of multiple database entities, obtaining audio data associated with the database entity, obtaining claims data associated with the database entity, generating a feature vector input according to the audio data and the claims data, processing, by the machine learning model, the feature vector input to generate the condition likelihood output, determining whether the condition likelihood output is greater than a specified likelihood threshold, and assigning the database entity to an identified condition subset of the multiple database entities in response to determining that the condition likelihood output is greater than the specified likelihood threshold. The method includes, for each database entity in the identified condition subset, transforming a user interface to display the condition likelihood output associated with the database entity.

In other features, memory hardware is configured to store multiple machine learning models each associated with a different one of multiple condition classification types, the method includes identifying one of the multiple machine learning models according to a specified condition prediction type, and processing the feature vector input includes processing the feature vector input using the selected machine learning model. In other features, the machine learning model includes a historic machine learning model, and training the historic machine learning model includes obtaining call transcription data associated with the multiple audio data entries, processing the call transcription data to using at least one of keyword and natural language processing to generate processed transcription input features, parsing the multiple audio data entries to define individual words, processing the defined individual words to generate processed audio input features, and supplying a training feature vector input to the machine learning model to train the machine learning model, wherein the training feature vector input includes the processed transcription input features and the processed audio input features.

In other features, the processed audio input features include at least one of an intensity of the audio waveform, a fundamental frequency, a formant frequency, Mel Frequency Cepstrum Coefficients (MFCCs), a glottal flow, a jitter value, a zero crossing value, a trailing intensity, and a white space length. In other features, wherein the machine learning model comprises a raw audio model, and the raw audio model comprises a one-dimensional convolution layer which receives an input of multiple frames, wherein the convolution layer includes multiple filters, a recurrent layer which uses the input from the convolution layer to identify temporal dependence through use of a Long Short-Term Memory (LSTM) layer, and a final layer that maps the convolution layer and the recurrent layer to a final output.

In other features, the machine learning model comprises a processed audio model, and training the processed audio model includes separating the multiple audio data entries into temporal frames with overlap, for each temporal frame, obtaining multiple processed audio features, wherein the multiple processed audio features include at least one of log-Mel bank features associated with the frame, Mel Frequency Cepstrum Coefficients associated with the frame, MFCC summary statistics associated with the frame, and MFCC difference values between a current temporal index and a prior temporal index, and supplying a training feature vector input to the processed audio model to train the processed audio model, wherein the training feature vector input includes the multiple processed audio features. In other features, the specified condition associated with one of the multiple historical database entities includes at least one of a post-partum depression medical condition, an anxiety medical condition, a drug addiction medical condition, a Parkinson's disease medical condition, and a respiratory disorder medical condition.

In other features, the method includes identifying a date associated with each of the multiple audio data entries and the multiple claims data entries, determining a date associated with a condition of each of the multiple historical database entities, based at least in part on the dates associated with the multiple claims data entries, and building a training dataset for training the machine learning model, wherein each audio data entry included in the training dataset has a date that is within a specified time window of the determined date associated with the condition of a corresponding one of the multiple historical database entities. In other features, training the machine learning model includes obtaining multiple social media data entries each associated with one of the multiple historical database entities, and generating one or more social media input features based on the multiple social media data entries, wherein the historical feature vector inputs include the one or more social media input features.

In a feature of the present disclosure, the prediction likelihood output can be a probabilistic output (e.g., a calculation result, an approximation range result, an estimate range result, or the like) associated with a given model. As this output is probabilistic, the prediction likelihood output can be described and represented as a distribution or associated characteristics of that distribution including variances quartiles, and intervals.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system for automated processing of audio waveform database entries using machine learning models.

FIG. 2 is a message sequence chart illustrating example interactions between components of the system of FIG. 1.

FIG. 3 is a flowchart depicting an example process for training a machine learning model to process audio waveform database entries.

FIGS. 4A and 4B are graphical representations of example recurrent neural networks for generating machine learning models for automated processing of audio waveform database entries.

FIG. 5 is a graphical representation of layers or an example long short-term memory (LSTM) machine learning model.

FIGS. 6A and 6B are graphical representations of example timelines for obtaining audio waveform data for model training.

FIG. 7 is a flowchart depicting an example process for building a training dataset for automated detection of a post-partum depression condition.

FIG. 8 is a flowchart depicting an example process for training a historical machine learning model to predict patient conditions based on audio waveform data.

FIG. 9 is a graphical representation of an example artificial neural network for automated processing of raw waveform audio data.

FIG. 10 is a flowchart depicting an example process for training a machine learning model using processed audio data.

FIG. 11 is a flowchart depicting an example process for generating a condition prediction based on call data for a patient.

In the drawings, reference numbers may be reused to identify similar and/or identical elements.

### DETAILED DESCRIPTION

Artificial neural networks (ANNs), or any suitable machine learning models, may be used to identify various medical or behavioral conditions based on audio data. Example models may use audio data inputs to generate condition predictions, may combine audio data with other modeling techniques such as patient claims history, etc. The various conditions may include, but are not limited to, post-partum depression, anxiety, addiction, Parkinson's disease, and respiratory disorders.

In various implementations, the methods of building and training machine learning models may be flexible and adaptable for different types of labels. For example, models may utilize speech-to-text, a time series of raw waveform audio, features generated from the spectral domain, etc. Inputs to the model may be obtained from any suitable sources, such as calls between a customer service representative and a customer with a potential health condition.

In some embodiments, a healthcare payer or pharmacy benefit manager may augment a provider with assumptions on a current patient state, and obtain information directly from the provider and indirectly from pharmacy and claims data. This data may be used with machine learning models that combine audio (and possibly video) signals to predict medical and behavioral conditions for members.

For example, the machine learning models may be used to simultaneously aid a healthcare provider in determining a patient's current state, and can also be used to help detect potential medical conditions through alternative interactions such as consumer calls. The models may assist in reaching out to people who are not engaging with behavioral health services but are exhibiting symptoms that behavioral health services may help, such as depression, anxiety, substance abuse, etc. More generally, the models may be improved via partnerships with experts (e.g., providers) to create well trained models in clinical settings, which can be applied to a greater population via customer service calls, etc.

### Audio Waveform Database Entity Processing System

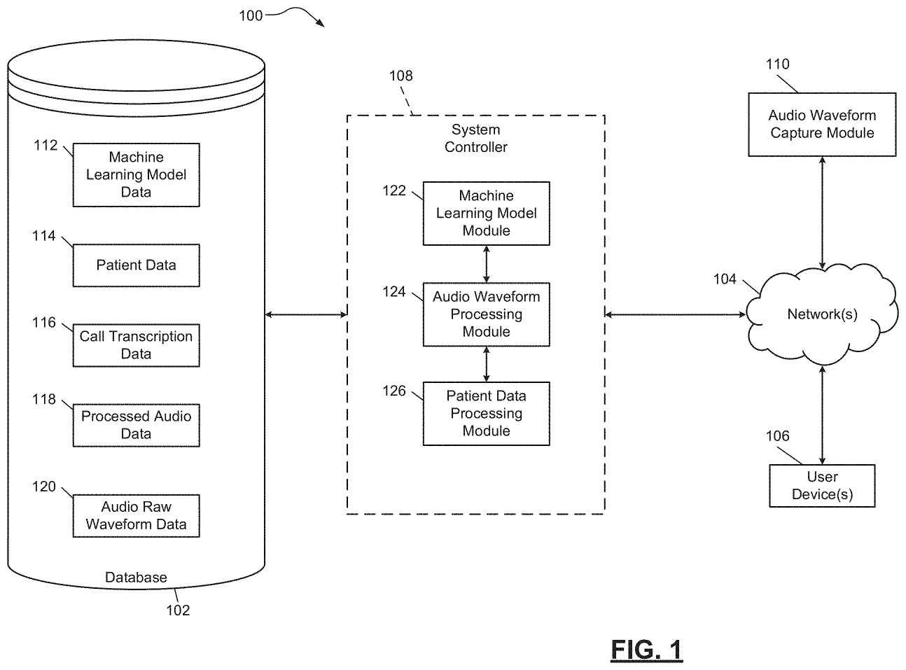

FIG. 1 is a functional block diagram of an example system 100 for automated processing of audio waveform database entities using machine learning models, which includes a database 102 (which may be referred to as a record database). While the system 100 is generally described as being deployed in a computer network system, the database 102 and/or components of the system 100 may otherwise be deployed (for example, as a standalone computer setup). The system 100 may include a desktop computer, a laptop computer, a tablet, a smartphone, etc.

As shown in FIG. 1, the database 102 stores machine learning model data 112, patient data 114, call transcription data 116, processed audio data 118, and audio raw waveform data 120. In various implementations, the database 102 may store other types of data as well. The machine learning model data 112, patient data 114, call transcription data 116, processed audio data 118, and audio raw waveform data 120 may be located in different physical memories within the database 102, such as different random access memory (RAM), read-only memory (ROM), a non-volatile hard disk or flash memory, etc. In some implementations, the machine learning model data 112, patient data 114, call transcription data 116, processed audio data 118, and audio raw waveform data 120 may be located in the same memory (such as in different address ranges of the same memory). In various implementations, the machine learning model data 112, patient data 114, call transcription data 116, processed audio data 118, and audio raw waveform data 120 may each be stored as structured or unstructured data in any suitable type of data store.

In various implementations, structured data may include organized stored data that is decipherable by machine learning algorithms, which may be stored in relational database(s) (e.g., SQL) that allow for fast input, searching and manipulation of structured data. Examples of structured data may include, but are not limited to, dates, names, addresses, credit card numbers, medical claim forms, input vectors for machine learning models, processed audio data, etc. In various implementations, unstructured data may include data that does not have a predefined data model, and may be managed in non-relational databases or data lakes to preserve data in raw form. Example unstructured data may include text, mobile activity, social media posts, Internet of Things (IoT) sensor data, audio data such as waveforms, etc. Data structures may refer to one or more storage locations that store structured and/or unstructured data, such as historical data structures that store data for training machine learning models (where the data in the historical data structures may or may not have been processed to generate input feature vectors). In various implementations, audio data, claims data, patient data, and other suitable data, may be referred to as one or more data entries, which may be stored in structured or unstructured formats.

The machine learning model data 112 may include any suitable data for training one or more machine learning models, such as historical data structures related to one or more of the patient data 114, call transcription data 116, processed audio data 118, and audio raw waveform data 120. The machine learning model data 112 may include historical feature vector inputs that are used to train one or more machine learning models to generate a prediction output, such as a prediction of a patient condition (for example, when audio data of a customer's speech from a call is indicative that the customer has a condition such as depression). The historical feature vector inputs may include the historical data structures which are specific to multiple historical database entities (such as multiple historical audio data waveforms that are associated with customers having one or more specified conditions). In an example embodiment, the prediction likelihood output is a probabilistic output (estimate) associated with a given model. As this output is probabilistic, the prediction likelihood output can be described and represented as a distribution or associated characteristics of that distribution including variances quartiles, and intervals. In an example embodiment, the prediction likelihood output is not deterministic, e.g., every time the model is run there may be a variation in the prediction likelihood output.

In various implementations, users may train a machine learning model by accessing the system controller 108 via the user device 106. The user device 106 may include any suitable user device for displaying text and receiving input from a user, including a desktop computer, a laptop computer, a tablet, a smartphone, etc. In various implementations, the user device 106 may access the database 102 or the system controller 108 directly, or may access the database 102 or the system controller 108 through one or more networks 104. Example networks may include a wireless network, a local area network (LAN), the Internet, a cellular network, etc.

The system controller 108 may include one or more modules for automated entity field correction. For example, FIG. 1 illustrates a machine learning model module 122, an audio waveform processing module 124, and a patient data processing module 126. The audio waveform processing module may be configured to receive audio waveforms from the audio waveform capture module 110, or the audio raw waveform data 120 stored in the database 102, and process the audio waveforms to generate processed audio data, such as the processed audio data 118 stored in the database 102.

The machine learning model module 122 may include one or more machine learning models, which may be trained based on, for example, the machine learning model data 112. The machine learning model module 122 may be trained to automatically process the one or more of the patient data 114, the call transcription data 116, the processed audio data 118, the audio raw waveform data 120, and audio received from the audio waveform capture module 110, such as by generating a prediction likelihood output that a customer on a phone call is currently experiencing a specified medical or behavioral condition.

The patient data processing module 126 may process the patient data 114 to determine prior medical history for a patient, risk scores for a patient, demographic information, etc. For example, the patient data processing module 126 may generate input feature vectors to be used by the machine learning model module 122 in combination with features generated by the audio waveform processing module, in order to generate a prediction likelihood output that a customer on a phone call (or other suitable speech sample) is experiencing a medical or behavioral condition.

As shown in FIG. 1, the system controller 108 may communicate with the audio waveform capture module 110 via the network(s) 104. For example, the audio waveform capture module 110 may capture audio waveforms associated with a patient's speech via a customer service call or other suitable speech source, and provide the audio waveforms to the system controller 108 for processing or use by the audio waveform processing module 124 and the machine learning model module 122.

Referring back to the database 102, the patient data 114 may include any suitable data records of patients and associated field values, such as a patient name, address, date of birth, phone number, diagnosis history, prior and current chronic conditions, health event risk scores, demographic information, and so on. The call transcription data 116 may include any suitable information regarding transcription of customer calls (such as patients or health insurance members making customer service calls). At least a portion of the call transcription data 116 may include processed data such as keywords and natural language processing (NLP) data. In various implementations, one or more modules of the system controller 108 may perform keyword processing, NLP algorithm(s), etc., on the call transcription data 116.

The processed audio data 118 may include any suitable audio features, which may be generated for use by the machine learning model module 122 based on the audio raw waveform data 120. Example processed audio features include, but are not limited to, an intensity of the audio waveform, a fundamental frequency, a formant frequency, Mel Frequency Cepstrum Coefficients (MFCCs), glottal flow, jitter, shimmer, a zero crossing value, a trailing intensity, and a white space length (e.g., from the proceeding word). The audio raw waveform data 120 may include, for example, raw audio waveforms obtained from customer calls that have not been processed to generate audio features.

In various implementations, the system 100 may obtain data for the machine learning model from any suitable sources. For example, images of a person may be obtained to determine their state, such as images sourced from social media accounts of the person. Written social media posts may be processed for text indictors.

Audio of a person's speech may be obtained from calls to a call center, and video may be obtained if the call is, e.g., a videoconferencing call, etc. Audio and/or video may also be obtained from a patient visit to a location, such as a treatment resource center, an addiction center, a medical clinic, a hospital visit, a doctor's office, etc. For example, a waiting room of a clinic or doctor's office may be recorded to identify potential conditions of visitors. Patients may be recorded under anesthesia to simulate the influence of narcotics.

In some embodiments, such as the examples described above, a standard questionnaire or phrases may be used to establish controls, such as persons under the influence of drugs and persons not under the influence of drugs. Captured data may be digitized to generate sample inputs for the models, and the data may be tokenized to keep smaller portions that are most relevant to generating condition predictions.

In various implementations, the architecture of the system 100 may include a mixture of cloud computing (such as using Amazon Web Services) and on-premises computation. For example, neural networks may be built using Tensorflow/Keras on AWS (which may be limited to audio data and transcription). Medical record features may be combined with the audio and transcription data on-premises.

For example, a Verint AWS bucket may be connected with AWS R Servers, that supply to a CSV AWS bucket. A scheduled aggregation script may be run on the CSV AWS bucket data, which provides analysis and modeling on-premises. In various implementations, an R script may look for unprocessed Verint data, write empty CSV data to an S3 bucket (lock), and convert to a useable format such as FFMPEG. The R script may segment audio, extract features from the segments, write the output to an S3 bucket, and erase audio data.

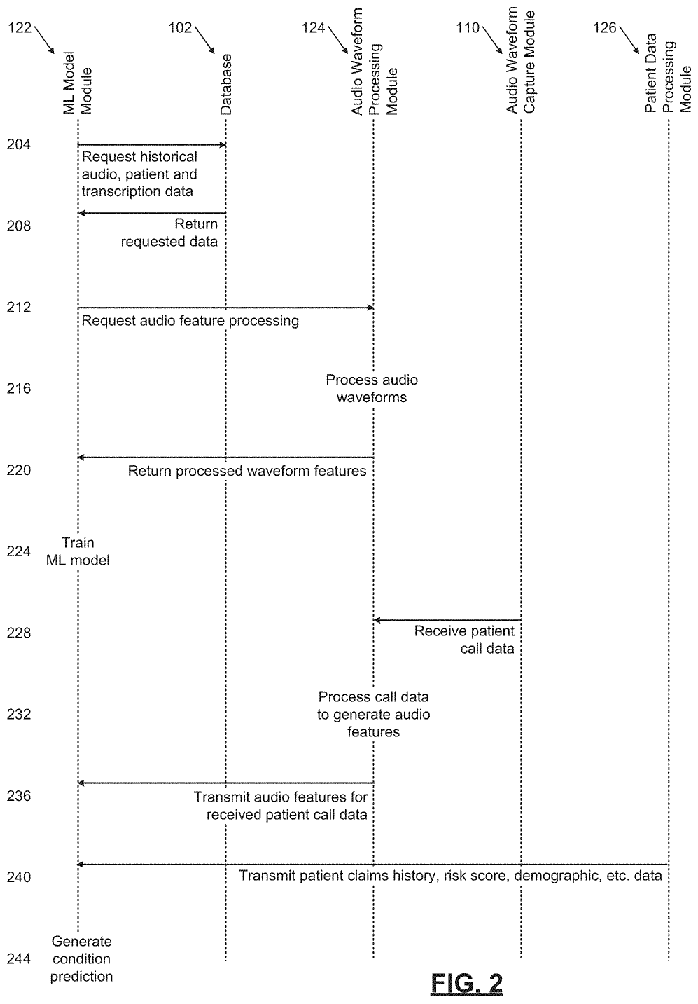

FIG. 2 is a message sequence chart illustrating example interactions between the database 102, the machine learning model module 122, the audio waveform processing module 124, the audio waveform capture module 110, and the patient data processing module 126. At line 204, the machine learning model module 122 requests historical audio, patient and/or call transcription data from the database 102.

For example, the machine learning model module 122 may request historical data structures from one or more of the patient data 114, the call transcription data 116, the processed audio data 118, and the audio raw waveform data 120. At line 208, the database 102 returns the requested historical data to the machine learning model module 122. The machine learning model module 122 requests the audio waveform processing module 124 to perform audio feature processing on the appropriate portions of the obtained historical data, at line 212.

The audio waveform processing module 124 then processes the audio waveforms at line 216, such as by generating one or more feature vector values similar to the example values listed above for the processed audio data 118. The processed waveforms are returned to the machine learning model module 122 at line 220, and the machine learning model module 122 then trains a machine learning model using the obtained data, at line 224. Any suitable machine learning model may be used, as described further below. For example, the machine learning model module 122 may train a historic model that utilizes generated features to predict the occurrence of a medical condition, may train an artificial neural network (ANN) to predict the occurrence of a medical condition using a time-series raw waveform audio data input, may train an ANN to predict the occurrence of a medical condition using a time-series of pre-processed audio data, etc.

At line 228, the audio waveform capture module 110 transmits audio waveform data for a received patient call to the audio waveform processing module 124. For example, the audio waveform capture module 110 may capture audio waveforms from a call between a health insurance member and a customer service representative. The audio waveform processing module then processes the received call data to generate audio features, at line 232.

At line 236, the audio waveform processing module 124 transmits the processed audio features to the machine learning model module 122. At line 240, the patient data processing module 126 transmits patient claims data, risk score data, demographic data, etc., to the machine learning model module 122. The transmitted data may include at least a portion of the patient data 114, which may be associated with a caller identified in the patient call data received at 228. The machine learning model module 122 then generates a condition prediction of a likelihood that the identified patient associated with the audio data is experiencing a specified medical condition, at line 244.

The machine learning model module 122 may process the audio data to look for characteristics in at least one change in the audio of a person's speech or a group of people's speech. The machine learning model module 122 may examine the speech data to identify changes in dysphonia over time as a function of a patient's health status. The health status may be determined from medical claim data, predicted from data related to the person, e.g., social media, credit data, geographic data, combinations thereof, and the like. The machine learning model module 122 may also take into account whether a patient is a smoker or a non-smoker. In some implementations, smoking status of the patient is taken into account as a factor in the machine learning model module 122.

In an example implementation, the machine learning model module 122 examines a change in the audio for a same person or a specific group of people, for a change in the jitter, shimmer and harmonic-to-noise compared to a control measurement. In example measurement of normalized acoustic analytical data for speech for females, vowels /a/and /é/ had average measures of: f₀ 205.82 Hz and 206.56 Hz; jitter of 0.62% and 0.59%; shimmer of 0.22 dB and 0.19 dB; PHR of 10.9 dB and 11.04 dB, respectively; for males, vowel /a/and /é/ had average measures of: f₀ 119.84 Hz and 118.92 Hz; jitter of 0.49% and 0.5%; shimmer of 0.22 dB and 0.21 dB; HNR 9.56 dB and 9.63 dB, respectively. For both f₀ and NHR, female measures were significantly higher than their male counterparts. The machine learning model module 122 may use these values or similar measurements for a control group.

### Machine Learning Model Training

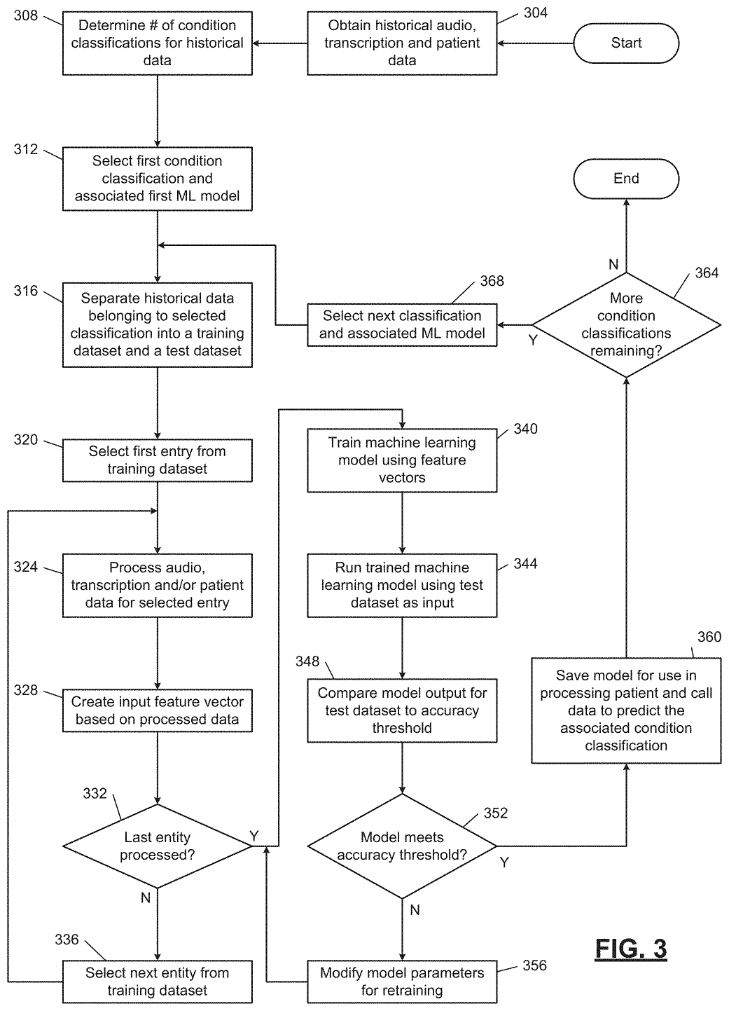

FIG. 3 illustrates an example process for training a machine learning module, which may be performed by, for example, the machine learning model module 122. Control begins at 304 by obtaining historical audio, transcription, and patient data. For example, historical data may be included in the machine learning model data 112, the patient data 114, the call transcription data 116, the processed audio data 118 and/or the audio raw waveform data 120, of the database 102.

At 308, control determines a number of condition classifications for the historical data. For example, different patient conditions may be specified, such as predicting whether the patient has post-partum depression, anxiety, addiction, Parkinson's disease, and respiratory disorders, and so on. Control selects the first condition classification and a machine learning model associated with the classification, at 312. Control then separates historical data belonging to the selected classification into a training dataset and a test dataset, at 316. For example, historical audio data, transcription data and patient data may be randomly divided where a portion of the data is used to train the model and another portion of the data is used to test the accuracy of the trained model.

At 320, control selects the first entry from the training dataset (such as an identified patient entry or patient entity in the database 102). Control then processes audio, transcription and/or patient data associated with the selected entry at 324. At 328, control creates an entity feature vector based on the processed audio, transcription and/or patient data. For example, keyword processing or NLP algorithms may be performed on the historical call transcription data, the raw audio waveform data may be processed to generate spectral domain features, etc.

Control determines whether the last entity has been processed at 332. For example, if more patient entities in the training dataset have not yet been processed to generate input feature vectors, control proceeds to 336 to select the next patient entity from the training dataset, and returns to 324 to process audio, transcription and/or patient data associated with the next selected entry. Once control determines at 332 that all patient entities within the training set have been processed to create input feature vectors, control proceeds to 340 to train the machine learning model using the feature vectors. For example, control may supply the input feature vectors as inputs to the machine learning model associated with the selected condition classification type. The machine learning model may generate a condition likelihood prediction output, indicative of a likelihood that the patient is experiencing the medical condition associated with the machine learning model. In an example embodiment, the condition likelihood prediction output is a probabilistic output (estimate) associated with a given model. As this output is probabilistic, the prediction likelihood output can be described and represented as a distribution or associated characteristics of that distribution including variances quartiles, and intervals.

At 344, control runs the trained machine learning model using the test dataset as the input (which may include creating input feature vectors for each patient entity in the test dataset). Control then compares the model output for the test dataset to an accuracy threshold at 348. For example, any suitable threshold may be used that is indicative of a desired accuracy of condition likelihood output predictions by the machine learning model, such as at least 50% correct patient condition likelihood determinations, at least 90% correct patient condition likelihood determinations, and so on.

If control determines at 352 that the output of the trained model on the test dataset does not meet the specified accuracy threshold, control modifies the model parameters for retraining at 356, and then returns to 340 to retrain the machine learning model using the training dataset input feature vectors with the modified model parameters. For example, hyper parameters of the machine learning model may be tuned to increase the accuracy of the model output on the training dataset.

Once control determines at 352 that the model output meets a specified accuracy threshold, control proceeds to 360 to save the trained model for use in processing other patient and call data to predict whether a speaker is experiencing the condition associated with the machine learning model. For example, if a machine learning model has been trained to predict a likelihood that the speaker has depression, the trained model may be stored to predict a likelihood that future callers have depression.

Control then determines at 364 whether more condition classifications are remaining. For example, if the number of condition classifications is determined at 308 to include three condition types, and machine learning models have been trained for the first two document types, control proceeds to 368 to select the next classification type associated with another machine learning model. Control then separates historical data belonging to that classification type into a training dataset and a test dataset at 316. Once control determines that there are no more condition classification types remaining for model training at 364, the process ends.

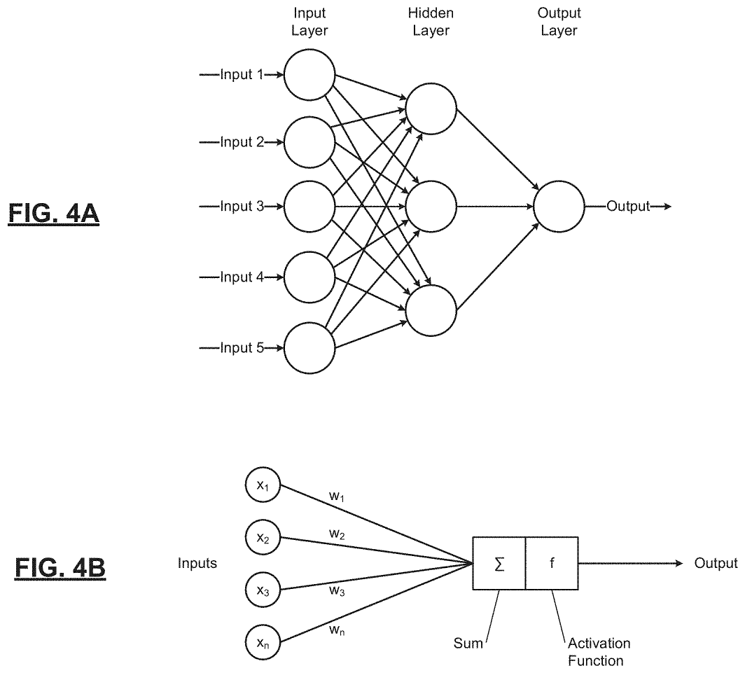

FIGS. 4A and 4B show an example of a recurrent neural network used to generate models such as those described above with reference to FIG. 1, using machine learning techniques. Machine learning is a method used to devise complex models and algorithms that lend themselves to prediction (for example, health plan customer predictions). The models generated using machine learning, such as those described above with reference to FIG. 1, can produce reliable, repeatable decisions and results, and uncover hidden insights through learning from historical relationships and trends in the data.

The purpose of using the recurrent neural-network-based model, and training the model using machine learning as described above with reference to FIG. 3, may be to directly predict dependent variables without casting relationships between the variables into mathematical form. The neural network model includes a large number of virtual neurons operating in parallel and arranged in layers. The first layer is the input layer and receives raw input data. Each successive layer modifies outputs from a preceding layer and sends them to a next layer. The last layer is the output layer and produces output of the system.

FIG. 4A shows a fully connected neural network, where each neuron in a given layer is connected to each neuron in a next layer. In the input layer, each input node is associated with a numerical value, which can be any real number. In each layer, each connection that departs from an input node has a weight associated with it, which can also be any real number (see FIG. 4B). In the input layer, the number of neurons equals number of features (columns) in a dataset. The output layer may have multiple continuous outputs.

The layers between the input and output layers are hidden layers. The number of hidden layers can be one or more (one hidden layer may be sufficient for most applications). A neural network with no hidden layers can represent linear separable functions or decisions. A neural network with one hidden layer can perform continuous mapping from one finite space to another. A neural network with two hidden layers can approximate any smooth mapping to any accuracy.

The number of neurons can be optimized. At the beginning of training, a network configuration is more likely to have excess nodes. Some of the nodes may be removed from the network during training that would not noticeably affect network performance. For example, nodes with weights approaching zero after training can be removed (this process is called pruning). The number of neurons can cause under-fitting (inability to adequately capture signals in dataset) or over-fitting (insufficient information to train all neurons; network performs well on training dataset but not on test dataset).

Various methods and criteria can be used to measure performance of a neural network model (such as for the model test result evaluation at 348 in FIG. 3). For example, root mean squared error (RMSE) measures the average distance between observed values and model predictions. Coefficient of Determination (R²) measures correlation (not accuracy) between observed and predicted outcomes (for example, between trained model outputs and actual outputs of the historical testing data from the machine learning model data 112). This method may not be reliable if the data has a large variance. Other performance measures include irreducible noise, model bias, and model variance. A high model bias for a model indicates that the model is not able to capture true relationship between predictors and the outcome. Model variance may indicate whether a model is stable (a slight perturbation in the data will significantly change the model fit).

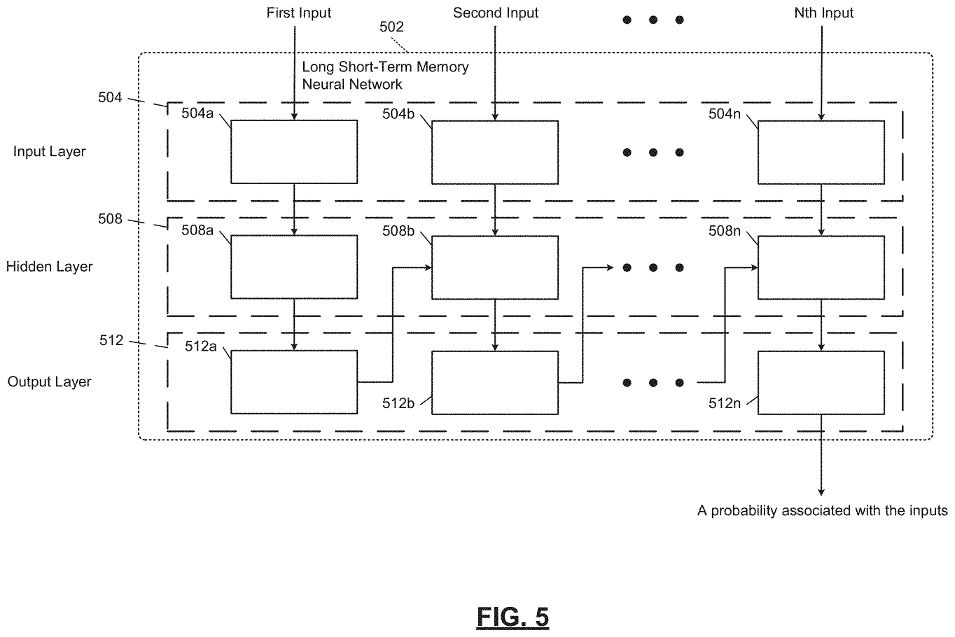

FIG. 5 illustrates an example of a long short-term memory (LSTM) neural network used to generate models such as those described above with reference to FIG. 3, using machine learning techniques. Machine learning is a method used to devise complex models and algorithms that lend themselves to prediction (for example, predicting entity field values in scanned document text of a prescription fill request). The models generated using machine learning, such as those described above with reference to FIG. 3, can produce reliable, repeatable decisions and results, and uncover hidden insights through learning from historical relationships and trends in the data.

The purpose of using the recurrent neural-network-based model, and training the model using machine learning as described above with reference to FIG. 3, may be to directly predict dependent variables without casting relationships between the variables into mathematical form. The neural network model includes a large number of virtual neurons operating in parallel and arranged in layers. The first layer is the input layer and receives raw input data. Each successive layer modifies outputs from a preceding layer and sends them to a next layer. The last layer is the output layer and produces output of the system.

FIG. 5 is a functional block diagram of a generic example LSTM neural network 502. The generic example LSTM neural network 502 may be used to implement the machine learning model trained by the process of FIG. 3, and various implementations may use other types of machine learning networks. The LSTM neural network 502 includes an input layer 504, a hidden layer 508, and an output layer 512. The input layer 504 includes inputs 504a, 504b . . . 504n. The hidden layer 508 includes neurons 508a, 508b . . . 508n. The output layer 512 includes outputs 512a, 512b . . . 512n.

Each neuron of the hidden layer 508 receives an input from the input layer 504 and outputs a value to the corresponding output in the output layer 512. For example, the neuron 508a receives an input from the input 504a and outputs a value to the output 512a. Each neuron, other than the neuron 508a, also receives an output of a previous neuron as an input. For example, the neuron 508b receives inputs from the input 504b and the output 512a. In this way the output of each neuron is fed forward to the next neuron in the hidden layer 508. The last output 512n in the output layer 512 outputs a probability associated with the inputs 504a-504n. Although the input layer 504, the hidden layer 508, and the output layer 512 are depicted as each including three elements, each layer may contain any number of elements.

In various implementations, each layer of the LSTM neural network 502 must include the same number of elements as each of the other layers of the LSTM neural network 502. For example, historical patient data may be processed to create the inputs 504a-504n. The output of the LSTM neural network 502 may represent a likelihood that a patient is experiencing a specified medical condition.

In some embodiments, a convolutional neural network may be implemented. Similar to LSTM neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 504a is connected to each of neurons 508a, 508b . . . 508n.

In various implementations, each input node in the input layer may be associated with a numerical value, which can be any real number. In each layer, each connection that departs from an input node has a weight associated with it, which can also be any real number. In the input layer, the number of neurons equals number of features (columns) in a dataset. The output layer may have multiple continuous outputs.

As mentioned above, the layers between the input and output layers are hidden layers. The number of hidden layers can be one or more (one hidden layer may be sufficient for many applications). A neural network with no hidden layers can represent linear separable functions or decisions. A neural network with one hidden layer can perform continuous mapping from one finite space to another. A neural network with two hidden layers can approximate any smooth mapping to any accuracy.

### Database Entity Condition Prediction

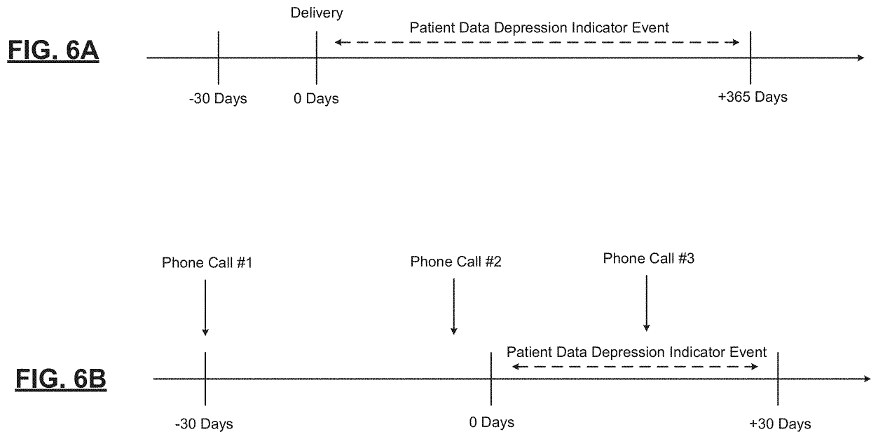

FIGS. 6A and 6B illustrate example timelines for obtaining audio waveform data for model training. For example, FIG. 6A illustrates a timeline for determining whether a patient (sometimes referred to as a database entity) is experiencing post-partum depression. Although post-partum depression is illustrated in the example of FIG. 6A, other suitable conditions may be used in other implementations as described further herein.

According to studies, post-partum depression affects 1 out of 10 women in the United States, and is an under diagnosed population. In one example implementations, the system 100 may have historical medical claims data for approximately 75,000 during a one year period. FIG. 6A illustrates that data is tracked up to 365 days after delivery to determine whether a patient is experiencing post-partum depression, although other suitable time windows may be used in other implementations.

As shown in FIG. 6A, a patient may be considered to have experienced post-partum depression if at least one patient data depression indicator event occurs within the 365 day time window after delivery. One or more modules of the system controller 108 may determine the date of a delivery based on a recorded pregnancy or delivery identifier, or an event code in medial data of a patient. The occurrence of depression may be detected based on any suitable data records, such as prescription claims, behavioral claims, medical claim, etc. Each event is associated with a date, which allows the system controller 108 to determine whether the depression occurrence happened during the post-partum time window.

FIG. 6B illustrates an example timeline for determining whether a specific phone call is likely to be associated with the patient experiencing the post-partum depression condition. For example, a phone call within a specified window of the depression event occurrence (such as 30 days after or 30 days before the depression event occurrence) may be identified as having the strongest likelihood of exhibiting audio features associated with the depression occurrence.

In the example of FIG. 6B, Phone Calls #2 and #3 may have a higher likelihood of including audio features associated with the post-partum depression condition because they are closer to the depression event occurrence than Phone Call #1. Therefore, the timeline of FIG. 6A can be used to determine whether a patient should be classified as having post-partum depression or not for the training data, and the timeline of FIG. 6B may be used to determine which phone calls are the most likely to include audio features associated with the post-partum depression condition.

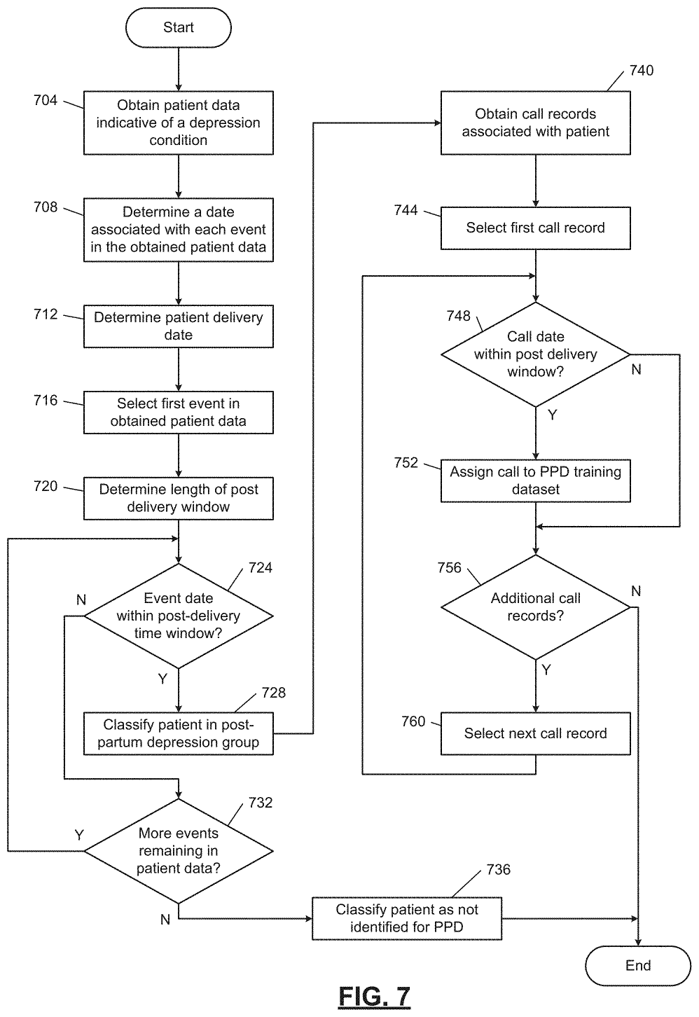

FIG. 7 illustrates an example process for building a training dataset for automated detection of a post-partum depression condition. In various implementations, the process of FIG. 7 may be performed by one or more modules of the system controller 108 of FIG. 1. At 704, control begins by obtaining patient data indicative of a depression condition (e.g., based on claims data, pharmacy data, etc.).

At 708, control determines a date associated with each event in the obtained patient data. For example, control may determine the date of each medical claim, each pharmacy fulfillment, etc. Control then determines a patient delivery date at 712. This may be determined by hospital records, claims data, etc. At 716, control selects a first event in the patient data.

Control determines a length of a post delivery time window at 720. For example, control may compare dates of depression indicative events to a time period of 365 days after delivery, six months after delivery, 90 days after delivery, or any other suitable time period.

At 724, control determines whether the selected event date is within the post-delivery time window. For example, control may determine whether the date associated with the depression indicative event is less than 365 days after the delivery date. If so, control proceeds to 728 to classify the patient in a post-partum depression group.

If the depression indicative event is not within the post-delivery time window, such as before delivery or more than 365 days after delivery, control proceeds to 732 to determine whether there are any more events remaining in the patient data. If not, control proceeds to 736 to classify the patient as not being identified for post-partum depression based on the event data associated with the patient. If control determines at 732 that there is at least one more event indicative of a depression condition associated with the patient, control returns to 724 to determine whether the date of the selected event is within the post-delivery time window.

When the patient is classified in the post-partum depression group at 728, control proceeds to 740 to obtain call records associated with the patient. The call records may include audio samples of patient speech, such as the patient calling to speak with a customer service representative or a doctor or other medical professional. At 744, control selects a first call record.

At 748, control determines whether a date of the selected call is within the post-delivery time window. If so, control assigns the call to a post-partum depression training dataset at 752. For example, if the patient has been classified in the post-partum depression group, and the selected call record is within a time window of the identified depression condition, there is a higher likelihood that the patient will have exhibited depression indicators in the patient's speech during the call.

After assigning the call to the post-partum depression dataset at 752, or if control determines at 748 that the date of the selected call was not within the post-delivery window, control proceeds to 756 to determine whether there are any additional call records associated with the patient. If so, control selects the next call record at 760 and returns to 748 to determine whether the date of the selected call is within the post-delivery date window. If there are no more call records associated with the patient at 756, control ends the process.

As mentioned above, post-partum depression is one example of a medical condition that may be predicted by example machine learning models described herein. The machine learning models may also be trained to detect other medical conditions (which may include behavioral conditions, mental health conditions, etc.).

For example, a machine learning model may be trained to predict a likelihood that a person is currently experiencing addiction. In this case, the model may be trained to identify one or more features in audio data of the member's speech that is associated with addiction, such as slurs in the speech, mispronunciation, pauses, forgetting basic words, difficulty in completing sentences, repetition of words or phrases, etc. The model may compare similar statements of one person against their own prior statements, or against similar statements by other people.

In various implementations, a machine learning model may be trained to identify phrases that are specific to members that may be experiencing drug addiction. For example, phrases or slang that are specific to using certain drugs, such as “I need a fix,” or “getting well,” etc., may be identified by a machine learning model. Such phrases may be particularly useful if they are not typically spoken by non-addicts. If audio inputs to the model include the specified phrases, the machine learning model may output a higher likelihood that the speaker is addicted to drugs.

As another example, a machine learning model may be trained to predict a likelihood of a depression condition. In this case, the model may be trained to look for specific tone in audio data of a member's speech, for a specific choice of words or phrases (such as cussing, slang, changes in word choice over time, etc.), pauses when speaking, a speed of the speech, or any other suitable features that have been highlighted as indicative of depression in published research studies. The model may be tuned to account for local slang based on a location of the member. In some cases, awkward pauses detected in the audio data may be indicative of medical conditions. In various implementations, the models may be used with other detector inputs to generate condition predictions, such as cough detectors, HIV detectors, etc.

In various implementations, a machine learning model may be trained to detect emphysema. For example, the machine learning model may be trained to identify a cough in sample audio data from a member, or a particular type of cough. The model may be trained to detect hoarseness in the speaker's voice. If the model is trained based on coughs, hoarseness, etc. from known emphysema patients, the model may more accurately predict a likelihood that another speaker has emphysema based on audio samples.

Outputs of the models may be used in various ways, such as to determine whether a person is abusing drugs. If the model generates a likelihood output indicative that a person is abusing drugs, additional audio samples may be obtained for that person to determine whether the person is suffering from addiction. For example, the system 100 may look at claims related to addiction, and then call the member to generate additional audio data for building the model.

In various implementations, outputs of the model may be supplied to other healthcare systems, such as Cigna's Health Connect 360 system. This may allow condition predictions to be supplied to healthcare providers so they can offer better medical care for patients. The condition prediction outputs may be supplied to a system that provides automated interventions, such as communicating with the patient to seek treatment or communicating with a healthcare provider to check on possible medical issues with a patient.

In some cases, dates of events may be used to determine when a condition started, in order to identify more relevant audio data around the condition date. For example, social media photographs could be used to identify when a member has an addiction problem. Social media data for a member could be combined with claims data to build a machine learning model.

Tokenizing audio data could provide a date index for recovery and relapse, which may have different timeframes. In the post-partum depression example, a woman may experience post-partum depression for one child but not another, and the machine learning models may differentiate between the different timelines based on dated events in the patient data. The models may be particularly useful for behavioral or mental health issues that are difficult to diagnose, e.g., because there may not be a single test that can be administered similar to respiratory diseases, etc.

As another example, a machine learning model may be trained to detect speech pattern changes associated with hypoxia. For example, acute hypoxia patients may have unusual intervals or pauses between words that are not natural to their spoken language, and may use speech that has a different fundamental frequency. If a machine learning model predicts a likelihood of acute hypoxia based on a member's speech, the system may generate an alert for a medical emergency to treat the acute hypoxia.

Conditions such as hypoxia and others that can change over time may be monitored by comparing baseline calls from earlier time frames to more current speech of a member. For example, the system may obtain a medical history including an audio record from a previous time, such as about one year ago, about five years ago, etc. The time period may or may not correspond to a prior medical diagnosis date. This earlier call can be used as a baseline to identify a person's speech prior to possible development of a medical condition. A more recent audio sample can be compared to the earlier baseline call data to determine whether a person's condition is worsening, or a new condition has developed.

For example, if the more recent call indicates a higher likelihood of acute hypoxia compared to the earlier baseline call, the system may generate an alert that the member appears to be in a more acute stage. In various implementations, the baseline and recent call data may be combined with other factors to generate targeted interventions. For example, if the more recent call data indicates the member has a new or worsening condition, and the claims data indicates that the member has not visited a doctor in an extended period of time (e.g., at least six months, at least one year, etc.), the system may provide a recommendation for the member to seek medical attention or transmit a notice to a medical care provider to reach out to the member to check on them.

### Machine Learning Model Examples

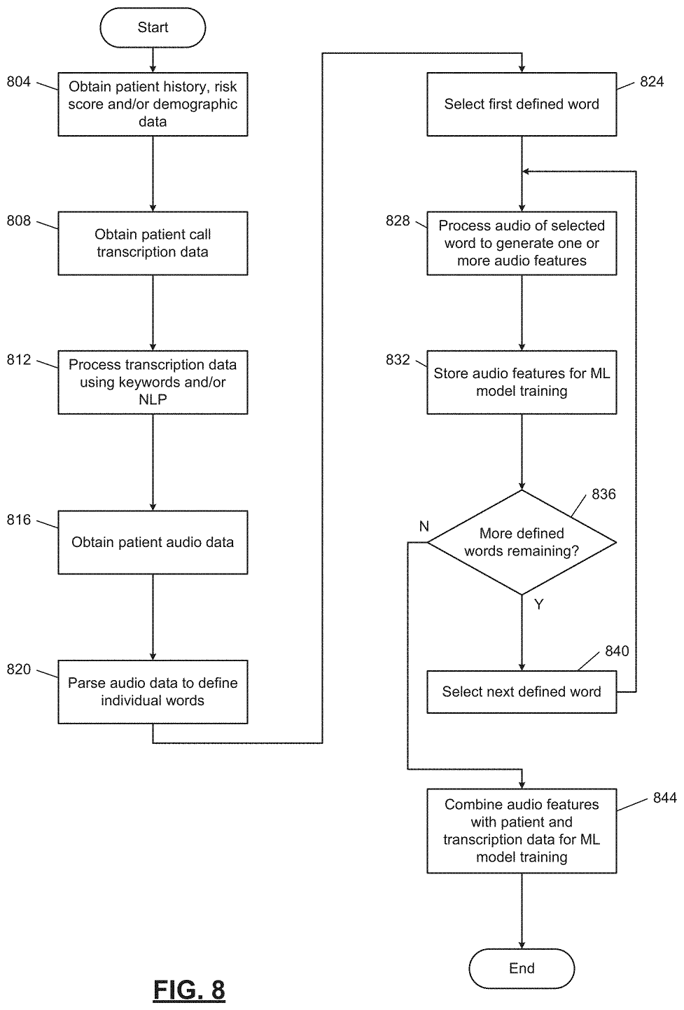

FIG. 8 is a flowchart depicting an example process for training a historical machine learning model to predict patient conditions based on audio waveform data. The process of FIG. 8 may be implemented by any suitable module, such as the machine learning model module 122 of FIG. 1.

At 804, control beings by obtaining patient history data, risk score data, demographic data, or any other suitable data associated with a patient. For example, the machine learning model module 122 may obtain historical data from the patient data 114 stored in the database 102.

Control then obtains patient call transcription data at 808, such as the call transcription data 116 stored in the database 102 that is associated with calls in which the patient was a participant. At 812, control processes the transcription data, such as by using keyword processing or natural language processing. For example, one or more modules of the system controller 108 may perform processing on the call transcription data 116.

At 816, control obtains patient audio data. The patient audio data may include any audio waveforms of speech of the patient, such as calls the patient was involved in. In various implementations, personally identifiable information may not be retained, in order to maintain privacy. Further, features may be restricted to be non-unique per member. In some cases, all raw audio data may be stored in a separate cloud storage bin, such as a separate s3 bin where the data was downloaded, scored, or trained on, and then the call audio is deleted. This approach may limit the surface area for privacy violations. In various implementations, all data may be deleted after a specified time period such as 180 days.

In some cases, there may be data quality issues in obtained audio files and associated metadata. For example, some calls may have incorrect metadata, where the person identified on the call was not actually part of the audio conversation. This may occur due to someone calling on behalf of the member, a health care provider or insurer discussing particular cases, etc. Control may screen obtained call data to remove incorrect records.

Control then parses the audio data to define individual words at 820. For example, the audio waveform processing module 124 of FIG. 1 may process the audio raw waveform data 120 stored in the database 102. In various implementations, each audio feature is defined for each word, where words are defined by continuous sections of waveform greater than a specified time period (such as 0.113 seconds with 5000 frames in 44.1 kHz wave file), separated by sections of background noise.

Background noise may be determined via a moving standard deviation applied to the normalized wave file. Continuous regions of length greater than a specified time period (such as 0.113 second), where the log of the moving standard deviation was less than, e.g., −10, may indicated areas of low activity. These areas or low activity may be identified as background noise or white space. In other implementations, other suitable time periods and log values may be used to identify words and background noise. The white space may be used to determine an increase in pauses in speech as an indicator of a disease state.

Control selects a first defined word at 824, then processes audio of the selected word to generate one or more audio features at 828. For example, the audio waveform processing module 124 may generate any suitable features from, e.g., the spectral domain, for the selected word. Example features include, but are not limited to, an intensity of the waveform, a fundamental frequency, a formant frequency, Mel Frequency Cepstrum Coefficients (MFCCs), glottal flow, jitter, a zero crossing value, a trailing intensity, and a white space length from a proceeding word. The audio features are then stored for training a historical machine learning model at 832.

At 836, control determines whether more defined words remain in the audio data (e.g., whether all words from a call record have been processed). If more words remain at 836, control selects the next defined word at 840 and returns to 828 to process audio of the selected word to generate one or more audio features. Once all words have been processed at 836, control proceeds to 844 to combine audio features with patient and transcription data for machine learning model training. In various implementations, each set of features may be trained against a target label, such as an ICD-10 diagnosis code or a diagnosis generated by business rules applied to claims. A separate holdout set may then be used to verify the performance of the model.

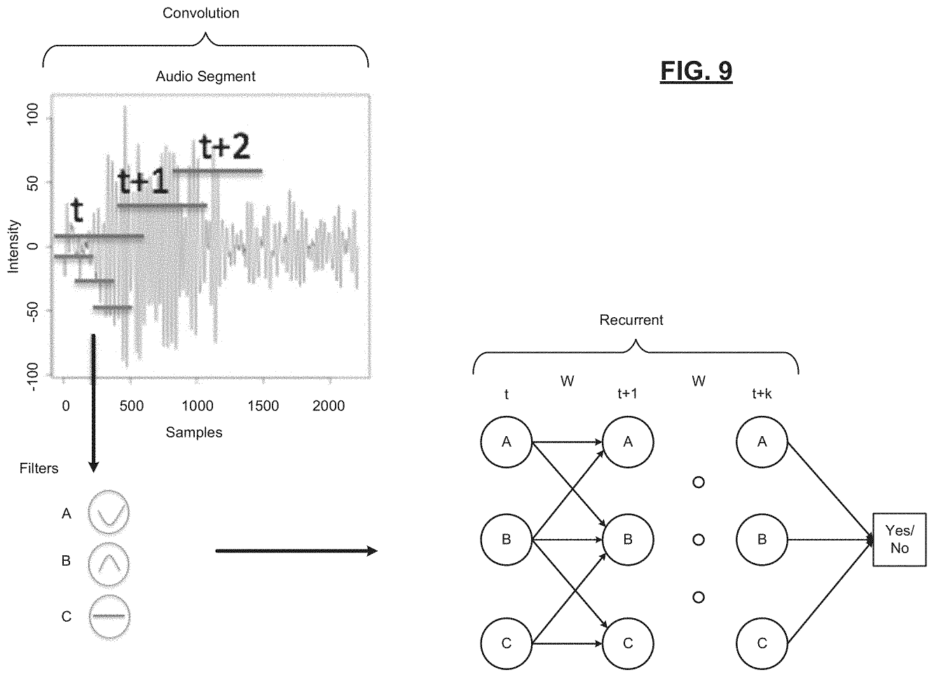

FIG. 9 is a graphical representation of an example artificial neural network (ANN) for automated processing of raw waveform audio data. The raw audio model is an ANN design that provides flexibility in identifying reoccurring time-lagged audio patterns. In various implementations, the model may be a variant of the compute library for deep neural networks (CLDNN) structure, used to construct an audio model using raw audio features as an approximation to traditional MFCC or log-Mel scale feature banks. In some cases, the model may not simply replace a transformation for converting speech to text, but instead approximate and discover more appropriate functions for the purpose of identification of health conditions such as depression and anxiety.

The example model illustrated in FIG. 9 is constructed using three ANN building blocks. First, the convolution layer is a 1-dimensional layer that takes an input of 600 frames (0.075 seconds), with an overlap of 100 frames between samples from the same audio file. This layer uses an initial convolution layer utilizing 80 filters and a kernel size of 10. The number of filters, kernel size, and input size may be adjusted based on application.

In various implementations, pooling may be performed (e.g., with a pool size of 9) to reduce the size of the input. Subsequent layers may be used with, for example, 160 filters and the same kernel size. Pooling may be performed after this layer as well. Additional convolution layers may be applied depending on computing resources and application.

The second layer (recurrent layer) illustrated in FIG. 9, uses the input from the convolution layer to identify temporal dependence. This temporal dependence may be obtained through the use of Long Short-Term Memory (LSTM) layers. Combined with the convolution layer, these layers determine the sub-sequences of the raw audio that best identify someone as having a particular medical condition.

The final layer illustrated in FIG. 9 maps the convolution and recurrent layers to the final output. The required depth of this layer may be dependent on both the complexity of the previous layers and the cardinality of the output. For a simple classification problem, it may be sufficient to have two nodes as part of a dense layer. The optimization method may be dependent on both the cardinality and scarcity of the categories of the output. For many cases, cross-entropy loss may be sufficient to obtain good results.

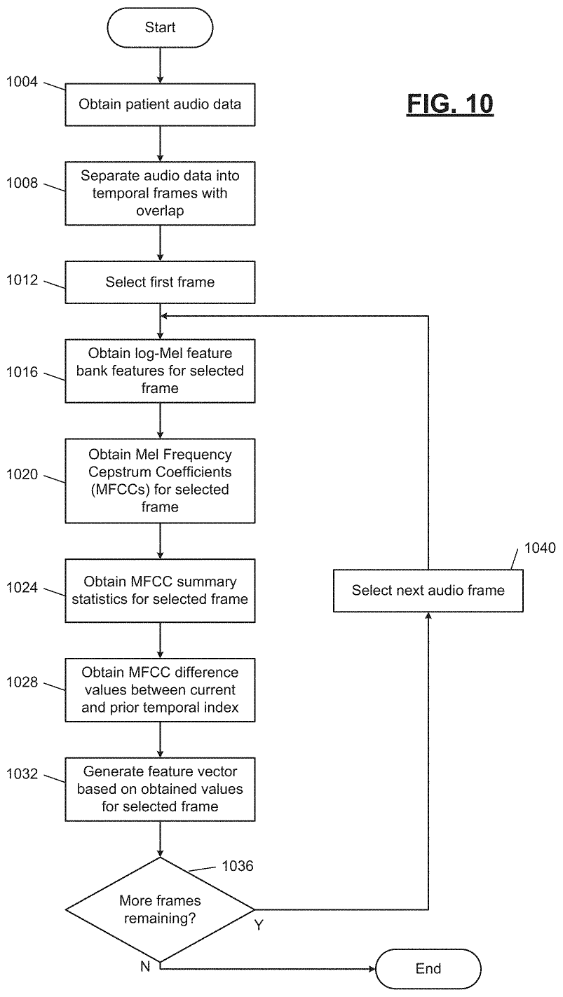

FIG. 10 is a flowchart depicting an example process for training a machine learning model using processed audio data. At 1004, control beings by obtaining patient audio data. Control then separates the audio data into temporal frames with overlap at 1008. For example, the audio data may be separated into 25 ms frames with 10 ms overlap (or any other suitable time periods).

At 1012, control selects the first frame. Control then obtains log-Mel feature bank features for the selected frame at 1016, and obtains Mel Frequency Cepstrum Coefficients for the selected frame at 1020. At 1024, control obtains MFCC summary statistics for the selected frame, and control obtains MFCC difference values between a current and prior temporal index at 1028.

Once all the audio processing features are obtained, control generates a feature vector for the selected frame at 1032 based on the obtained values. For example, in one implementation there may be 55 total features indexed by time, which include 13 MFCC features, 13 MFCC difference features, 3 MFCC summary statistic features, and 26 log-Mel feature bank features. In other implementations, other suitable combinations of features may be used.

At 1036, control determines whether more frames remain in the separated audio data. If so, control selects the next audio frame at 1040 and returns to 1016 to obtain the log-Mel feature bank features for the next selected audio frame. Once all frames have been processed at 1036, control ends the process. In various implementations, the output of the feature combination may be fed into a similar model as in the raw waveform example above, except using a two dimensional filter to accommodate the increase in dimension.

In various implementations, models may be mixed through the use of ensembles. For example, models may be mixed through conjugates, where the historic and ANN predictions can be viewed as both having normal distributions, where the distribution of the ANN's mean is dependent on the historic mean for a particular individual. Under this assumption, the estimate becomes a blend of both distributions with the following form:

$$= \frac{{σ}_{h}}{{σ}_{a} + {σ}_{h}} + \frac{{σ}_{a}}{{σ}_{a} + {σ}_{h}}$$

where [symbol] is the estimate for the individual, σₕ is the historic variance and [symbol] is the historic mean; σₐ would be the ANN variance, and [symbol] is the ANN mean. In various implementations, natural conjugates may be used to combine the historic and ANN approaches.

In various implementations, any suitable machine learning model arrangements may be used. For example, an XGBoost model using words may be implemented, a dense 3 layer neural network model may be used, a single layer LSTM (GRU) model or a multi-layer LSTM (GRU) (Single Class CLDNN) model may be used, etc. In some cases, medical data may be modeled using XGBoost with natural conjugates for ensembles.

Model training may be performed on a holdout dataset of, e.g., 100 calls identified as likely having post-partum depression, and 100 calls from individuals unlikely to have post-partum depression. Calls may be truncated to only cover a specified period of time, such as the first two minutes of conversation. In various implementations, models may be trained over a large number of audio files, such approximately 300,000 audio files. Some models may use 5-fold validation. In various implementations, modeling may retrain the same partitions between different modeling strategies and ensemble components.

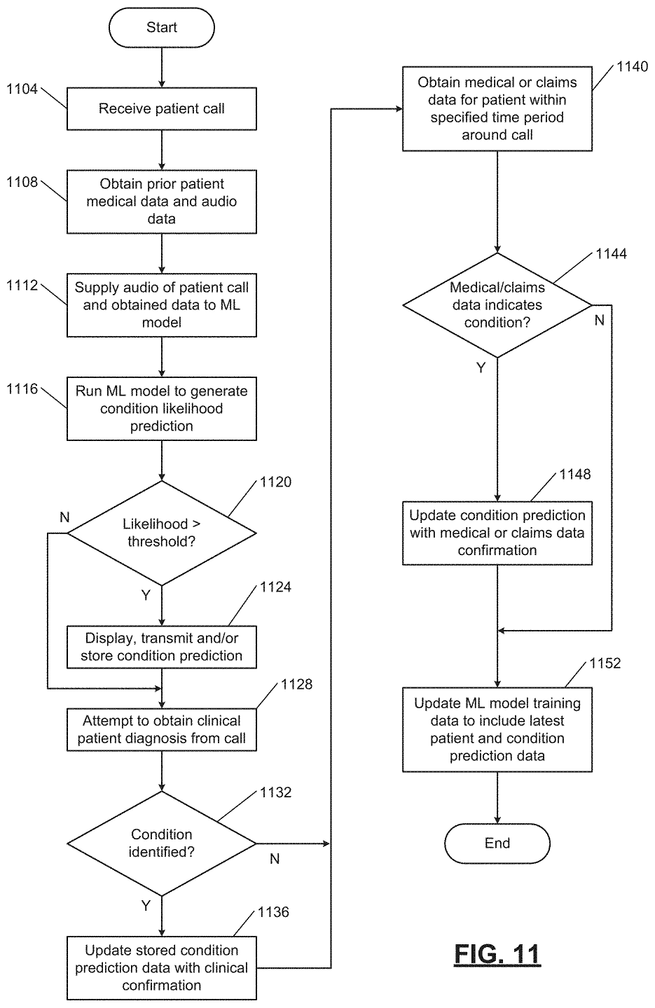

FIG. 11 is a flowchart depicting an example process for generating a condition prediction based on call data for a patient. At 1104, control begins by receiving a patient call (for example, via the audio waveform capture module 110 of FIG. 1). Control then obtains prior medical data and audio data associated with the patient, at 1108.

At 1112, control supplies the received audio of the patient call and the obtained data to a machine learning model. Control then runs the machine learning model at 1116 to generate a condition likelihood prediction output (e.g., a prediction of a likelihood that the patient has a specified condition based on the call data and the obtained patient records). For example, the machine learning model module 122 of FIG. 1 may receive the patient call audio data and the historical patient records as input to a machine learning model.

At 1120, control determines whether the generated likelihood output of the model is greater than a specified threshold value. Any suitable threshold value may be used, such as at least a 50% likelihood that the patient has a specified condition, a 90% likelihood that the patient has a specified condition, etc. If control determines that the likelihood output is greater than the threshold at 1120, control proceeds to 1124 to display, transmit and/or store the condition prediction. For example, control may store the condition prediction in medical records of the patient, may transform a user interface to display the condition prediction to a medical professional or administrator, may transmit an alert to a medical professional or directly to the patient, etc.

After displaying, transmitting, or storing the condition prediction at 1124, or control determines at 1120 that the prediction likelihood is less than a threshold value, control proceeds to 1128 to attempt to obtain a clinical diagnosis from the call. For example, if the call was with a medical provider and the medical provider records a diagnosis based on evaluation of the patient, control may obtain the diagnosis to confirm the prediction likelihood output. If control determines at 1132 that a clinical diagnosis identified the condition, control updates the stored condition prediction data with the clinical confirmation at 1136.

At 1140, control obtains medical or claims data for the patient within a specified time period around the call. For example, control may obtain medical claims data for 30 days prior to the call, control may wait 30 days after the call to obtain subsequent medical or prescription drugs claim data, etc. At 1144, control determines whether obtained medical or claims data indicates the predicted condition. If so, control updates the condition prediction data with the medical or claims data confirmation, at 1148. Control then proceeds to 1152 to update the machine learning model training data to include the latest patient and condition prediction data, which may include confirmations based on a clinical diagnosis or related claims data.

In various implementations, the machine learning models may be used in a clinical/behavioral setting where an expert is available. This may allow the models to be more quickly refined, where a feedback loop informs and corrects the model. Health care providers or payers may have access to pharmacy and claims data from a behavioral visit, allowing insight into the outcome of the visit, even if a condition was not recorded from the visit.

As described above, raw and processed audio data has value for identifying individuals with depression or other medical or behavioral conditions, with positive results even for lower quality audio data. Further, combination the data with audio records may substantially improve the ability to detect an individual with depression or other medical or behavioral conditions, creating a useful model prediction to inform the state of the individual.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In an example, a module can include electrical circuitry executing instructions, e.g., circuitry for data and instruction storage and processing circuitry to execute the instructions. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer system comprising:
memory hardware configured to store a machine learning model, historical feature vector inputs, and computer-executable instructions, wherein the historical feature vector inputs include historical data structures specific to multiple historical database entities, and wherein the historical data structures include multiple audio data entries and multiple claims data entries; and
processor hardware configured to execute the instructions, wherein the instructions include:
training the machine learning model with the historical feature vector inputs, including the multiple audio data entries and multiple claims data entries, to generate a condition likelihood output, wherein the condition likelihood output is indicative of a specified condition associated with one of the multiple historical database entities;
obtaining a set of multiple database entities;
for each database entity in the set of multiple database entities:
obtaining audio data associated with the database entity;
obtaining claims data associated with the database entity;
generating a feature vector input according to the audio data and the claims data;
processing, by the machine learning model, the feature vector input to generate the condition likelihood output;
determining whether the condition likelihood output is greater than a specified likelihood threshold; and
assigning the database entity to an identified condition subset of the multiple database entities in response to determining that the condition likelihood output is greater than the specified likelihood threshold; and
for each database entity in the identified condition subset, transforming a user interface to display the condition likelihood output associated with the database entity.

2. The system of claim 1, wherein:
the memory hardware is configured to store multiple machine learning models each associated with a different one of multiple condition classification types;
the instructions include, identifying one of the multiple machine learning models according to a specified condition prediction type; and
processing the feature vector input includes processing the feature vector input using the selected machine learning model.

3. The system of claim 1, wherein the machine learning model includes a historic machine learning model, and training the historic machine learning model includes:
obtaining call transcription data associated with the multiple audio data entries;
processing the call transcription data to using at least one of keyword and natural language processing to generate processed transcription input features;
parsing the multiple audio data entries to define individual words;
processing the defined individual words to generate processed audio input features; and
supplying a training feature vector input to the machine learning model to train the machine learning model, wherein the training feature vector input includes the processed transcription input features and the processed audio input features.

4. The system of claim 3, wherein the processed audio input features include at least one of an intensity of the audio waveform, a fundamental frequency, a formant frequency, Mel Frequency Cepstrum Coefficients (MFCCs), a glottal flow, a jitter value, a zero crossing value, a trailing intensity, and a white space length.

5. The system of claim 1, wherein the machine learning model comprises a raw audio model, and the raw audio model comprises:
a one-dimensional convolution layer which receives an input of multiple frames, wherein the convolution layer includes multiple filters;
a recurrent layer which uses the input from the convolution layer to identify temporal dependence through use of a Long Short-Term Memory (LSTM) layer; and
a final layer that maps the convolution layer and the recurrent layer to a final output.

6. The system of claim 1, wherein the machine learning model comprises a processed audio model, and training the processed audio model includes:
separating the multiple audio data entries into temporal frames with overlap;
for each temporal frame, obtaining multiple processed audio features, wherein the multiple processed audio features include at least one of log-Mel bank features associated with the frame, Mel Frequency Cepstrum Coefficients associated with the frame, MFCC summary statistics associated with the frame, and MFCC difference values between a current temporal index and a prior temporal index; and
supplying a training feature vector input to the processed audio model to train the processed audio model, wherein the training feature vector input includes the multiple processed audio features.

7. The system of claim 1, wherein the specified condition associated with one of the multiple historical database entities includes at least one of a post-partum depression medical condition, an anxiety medical condition, a drug addiction medical condition, a Parkinson's disease medical condition, and a respiratory disorder medical condition.

8. The system of claim 1, wherein the instructions include:
identifying a date associated with each of the multiple audio data entries and the multiple claims data entries;
determining a date associated with a condition of each of the multiple historical database entities, based at least in part on the dates associated with the multiple claims data entries; and
building a training dataset for training the machine learning model, wherein each audio data entry included in the training dataset has a date that is within a specified time window of the determined date associated with the condition of a corresponding one of the multiple historical database entities.

9. The system of claim 1, wherein training the machine learning model includes:
obtaining multiple social media data entries each associated with one of the multiple historical database entities; and
generating one or more social media input features based on the multiple social media data entries, wherein the historical feature vector inputs include the one or more social media input features.

10. The system of claim 1, wherein training the machine learning model includes:
comparing multiple condition likelihood outputs of the machine learning model to the historical data structures;
determining whether an accuracy of the comparison is greater than or equal to a specified accuracy threshold;
adjusting parameters of the machine learning model to retrain the machine learning model, in response to the accuracy of the comparison being less than the specified accuracy threshold; and
saving the machine learning model for use in generating condition likelihood outputs, in response to the accuracy of the comparison being greater than or equal to the specified accuracy threshold.

11. The system of claim 1, wherein training the machine learning model includes:
separating portions of the historical feature vector inputs into structured training data and structured test data;
training the machine learning model using the structured training data;
testing the trained machine learning model using the structured test data;
evaluating results of testing the trained machine learning model; and
saving the machine learning model for use in generating condition likelihood outputs that are probabilistic, in response to an accuracy of the evaluated results being greater than or equal to a specified accuracy threshold.

12. A computerized method for automated processing of audio waveform database entries using a machine learning model, the method comprising:
training a machine learning model with the historical feature vector inputs to generate a condition likelihood output, wherein the historical feature vector inputs include historical data structures specific to multiple historical database entities, wherein the historical data structures include multiple audio data entries and multiple claims data entries, and wherein the condition likelihood output is indicative of a specified condition associated with one of the multiple historical database entities;
obtaining a set of multiple database entities;
for each database entity in the set of multiple database entities:
obtaining audio data associated with the database entity;
obtaining claims data associated with the database entity;
generating a feature vector input according to the audio data and the claims data;
processing, by the machine learning model, the feature vector input to generate the condition likelihood output;
determining whether the condition likelihood output is greater than a specified likelihood threshold; and
assigning the database entity to an identified condition subset of the multiple database entities in response to determining that the condition likelihood output is greater than the specified likelihood threshold; and
for each database entity in the identified condition subset, transforming a user interface to display the condition likelihood output associated with the database entity.

13. The method of claim 12, wherein:
memory hardware is configured to store multiple machine learning models each associated with a different one of multiple condition classification types;
the method includes identifying one of the multiple machine learning models according to a specified condition prediction type; and
processing the feature vector input includes processing the feature vector input using the selected machine learning model.

14. The method of claim 12, wherein the machine learning model includes a historic machine learning model, and training the historic machine learning model includes:
obtaining call transcription data associated with the multiple audio data entries;
processing the call transcription data to using at least one of keyword and natural language processing to generate processed transcription input features;
parsing the multiple audio data entries to define individual words;
processing the defined individual words to generate processed audio input features; and
supplying a training feature vector input to the machine learning model to train the machine learning model, wherein the training feature vector input includes the processed transcription input features and the processed audio input features.

15. The method of claim 14, wherein the processed audio input features include at least one of an intensity of the audio waveform, a fundamental frequency, a formant frequency, Mel Frequency Cepstrum Coefficients (MFCCs), a glottal flow, a jitter value, a zero crossing value, a trailing intensity, and a white space length.

16. The method of claim 12, wherein the machine learning model comprises a raw audio model, and the raw audio model comprises:
a one-dimensional convolution layer which receives an input of multiple frames, wherein the convolution layer includes multiple filters;
a recurrent layer which uses the input from the convolution layer to identify temporal dependence through use of a Long Short-Term Memory (LSTM) layer; and
a final layer that maps the convolution layer and the recurrent layer to a final output.

17. The method of claim 12, wherein the machine learning model comprises a processed audio model, and training the processed audio model includes:
separating the multiple audio data entries into temporal frames with overlap;
for each temporal frame, obtaining multiple processed audio features, wherein the multiple processed audio features include at least one of log-Mel bank features associated with the frame, Mel Frequency Cepstrum Coefficients associated with the frame, MFCC summary statistics associated with the frame, and MFCC difference values between a current temporal index and a prior temporal index; and
supplying a training feature vector input to the processed audio model to train the processed audio model, wherein the training feature vector input includes the multiple processed audio features.

18. The method of claim 12, wherein the specified condition associated with one of the multiple historical database entities includes at least one of a post-partum depression medical condition, an anxiety medical condition, a drug addiction medical condition, a Parkinson's disease medical condition, and a respiratory disorder medical condition.

19. The method of claim 12, further comprising:
identifying a date associated with each of the multiple audio data entries and the multiple claims data entries;
determining a date associated with a condition of each of the multiple historical database entities, based at least in part on the dates associated with the multiple claims data entries; and
building a training dataset for training the machine learning model, wherein each audio data entry included in the training dataset has a date that is within a specified time window of the determined date associated with the condition of a corresponding one of the multiple historical database entities.

20. The method of claim 12, wherein training the machine learning model includes:
obtaining multiple social media data entries each associated with one of the multiple historical database entities; and
generating one or more social media input features based on the multiple social media data entries, wherein the historical feature vector inputs include the one or more social media input features.
